(19) United States
(12) Patent Application Publication
(10) Pub. No.: US 2025/0009278 A1
(43) Pub. Date: Jan. 9, 2025

(54) ELECTROANATOMICAL MAPPING DEVICE WITH INTERNAL ELECTRODE

(71) Applicant: Boston Scientific Scimed, Inc., Maple Grove, MN (US)

(72) Inventors: Prateek Mathur, Toronto (CA); Lauren Koon, Etobicoke (CA); Eduardo Moriyama, Richmond (CA); Charlene Leung, Scarborough (CA); Jackie Leung, Richmond Hill (CA)

(21) Appl. No.: 18/762,964

(22) Filed: Jul. 3, 2024

Related U.S. Application Data
(60) Provisional application No. 63/512,405, filed on Jul. 7, 2023.

(51) Int. Cl. A61B 5/367; A61B 5/287; A61B 5/343

(52) U.S. Cl. CPC A61B 5/367; A61B 5/287; A61B 5/343

(57) ABSTRACT

A medical system for creating a communication channel between two anatomical structures includes an outer member having a proximal portion and a distal portion, and a lumen extending from the proximal portion to the distal portion. At least a portion of the lumen includes an electrically conductive surface extending to a distal end of the distal portion. The system includes an inner member having a proximal portion and a distal portion. The inner member is configured to translate within the lumen and to deliver a therapy to target tissue. The inner member distal portion includes a conductive distal tip configured to electrically couple with the electrically conductive surface of the outer member. The inner member includes a proximal portion adapted to electrically couple the conductive distal tip to an auxiliary device.

### CROSS REFERENCE TO RELATED APPLICATIONS

The present application claims priority to U.S. Provisional Patent Application Ser. No. 63/512,405, filed Jul. 7, 2023, the entire disclosure of which is incorporated herein by reference.

### TECHNICAL FIELD

The present invention relates generally to methods and devices usable within the body of a patient. More specifically, the present invention is concerned with a device having an internal electrode that can be visualized using an Electroanatomical mapping (EAM) system.

### BACKGROUND

Electroanatomical mapping (EAM) is an increasingly prevalent technology useful during in vivo procedures. It enables physicians to identify anatomical regions of the heart and patterns of electrical activation. This is especially useful when treating arrhythmias. Devices that are compatible with EAM systems allow operators to localize them and more easily target specific regions for treatment, allowing for better workflow, better treatment efficacy, and shorter procedure time.

Typically, each EAM device requires a cable to connect to the EAM system. This can result in cumbersome manipulation of the device and a challenging workspace to manage with several cables for the procedure's devices.

Devices can interface with EAM systems with electrodes integrated on the surface of the device. The electrodes enable position tracking of the device on the EAM system via impedance measurements. However, embedded electrodes on the surface of elongated devices introduce transitions. These transitions increase the risk of causing injury when navigating through vasculature. In addition, transitions potentially impede the device's ability to traverse through tight openings, such as holes created by transseptal puncture or entering through a hemostasis valve.

Additionally, placing an electrode at the distal tip of an elongated device that has a lumen presents additional manufacturing challenges and the risk of exposed edges at the electrode interface is even greater. Ring electrodes on lumen devices typically are placed a few millimeters from the tip to properly secure it and provide a sufficiently smooth material transition.

### SUMMARY

Example 1 is a medical system for creating a channel between two anatomical structures includes an outer member having a proximal portion and a distal portion, and a lumen extending from the proximal portion to the distal portion. At least a portion of the lumen includes an electrically conductive surface extending to a distal end of the distal portion. The system includes an inner member having a proximal portion and a distal portion. The inner member is configured to translate within the lumen and to deliver a therapy to a target tissue. The inner member distal portion includes a conductive distal tip configured to electrically couple with the electrically conductive surface of the outer member. The inner member includes a proximal portion adapted to electrically couple the conductive distal tip to an auxiliary device.

Example 2 is the system of Example 1, wherein the outer member is a hypotube.

Example 3 is the system of Example 1, wherein the electrically conductive surface includes a coating.

Example 4 is the system of any of Examples 1-3, wherein the electrically conductive surface extends from the outer member proximal portion to the distal end of the distal portion.

Example 5 is the system of any of Examples 1-4, wherein the therapy delivered by the inner member is to puncture the target tissue.

Example 6 is the system of any of Examples 1-5, wherein the outer member includes a layer of insulation surrounding the conductive surface.

Example 7 is the system of any of Examples 1-6, wherein the auxiliary device is an EAM system adapted to identify and display a location of the distal end.

Example 8 is the system of any of Examples 1-7, wherein the outer member distal portion includes a taper.

Example 9 is the system of any of Examples 1-8, wherein the outer member distal portion includes an electrode.

Example 10 is the system of Example 9, wherein the electrode is formed by the electrically conductive surface.

Example 11 is the system of Example 9, wherein the electrode is flush with an outer surface of the outer member.

Example 12 is the system of any of Examples 1-11, wherein the inner member includes a proximal contact configured to electrically couple with the electrically conductive surface.

Example 13 is the system of Example 12, wherein the proximal contact includes an enlarged portion.

Example 14 is the system of Example 12, wherein the proximal contact includes a retrograde wire.

Example 15 is the system of any of Examples 1-14, wherein the outer member distal portion includes a distal tip, and the electrically conductive surface extends beyond the distal tip.

Example 16 is a medical system for creating a channel between two anatomical structures, such as crossing the atrial septum in a patient's heart, having an outer member including a proximal portion and a tapered distal portion. The outer member includes a lumen extending from the proximal portion to the tapered distal portion. At least a portion of the lumen includes an electrically conductive surface extending to a distal end of the tapered distal portion. The system includes an inner member having a proximal portion and a distal portion. The inner member is configured to translate within the lumen and to deliver a therapy to a target tissue. The inner member distal portion includes a conductive distal tip configured to electrically couple with the electrically conductive surface of the outer member. The inner member includes a proximal portion adapted to electrically couple the conductive distal tip to an auxiliary device

Example 17 is the system of Example 16, wherein the outer member is a hypotube.

Example 18 is the system of Example 16, wherein the electrically conducive surface includes a coating.

Example 19 is the system of Example 16, wherein the electrically conductive surface extends from the outer member proximal portion to the distal end of the tapered distal portion.

Example 20 is the system of Example 16, wherein the therapy delivered by the inner member is to puncture the target tissue.

Example 21 is the system of Example 16, wherein the outer member includes a layer of insulation surrounding the electrically conductive surface.

Example 22 is the system of Example 16, wherein the auxiliary device is an EAM system adapted to identify and display a location of the distal end.

Example 23 is the system of Example 16, wherein the outer member distal portion includes an electrode.

Example 24 is the system of Example 23, wherein the electrode is formed by the electrically conductive surface.

Example 25 is the system of Example 23, wherein the electrode is flush with an outer surface of the outer member.

Example 26 is the system of Example 16, wherein the inner member includes a proximal contact configured to electrically couple with the electrically conductive surface.

Example 27 is the system of Example 26, wherein the proximal contact includes an enlarged portion.

Example 28 is the system of Example 26, wherein the proximal contact includes a retrograde wire.

Example 29 is the system of Example 16, wherein the outer member distal portion includes a distal tip, and the electrically conductive surface extends beyond the distal tip.

Example 30 is a medical system for creating a channel between two structures, such as crossing the atrial septum in a patient's heart, including an outer member having a proximal portion and a distal portion. A lumen extends from the proximal portion to the tapered distal portion. A conductive surface is located along at least a portion of the lumen. The conductive surface extends to the tapered distal portion. A layer of insulation surrounds the conductive surface. The system includes an inner member having a proximal portion and a distal portion. The inner member is configured to translate within the lumen. The inner member distal portion includes a distal tip configured to electrically couple with the conductive surface and a proximal end configured to electrically couple with an EAM system.

Example 31 is the system of Example 30, wherein the outer member comprises a hypotube.

Example 32 is the system of Example 30, wherein the conducive surface includes a coating.

Example 33 is the system of Example 30, wherein the inner member includes a proximal contact configured to electrically couple with the conductive surface.

Example 34 is the system of Example 33, wherein the proximal contact includes an enlarged portion or a retrograde wire.

Example 35 is a method of performing a medical procedure including inserting an inner member into a lumen of an outer member, wherein the lumen includes a conductive surface. The procedure includes electrically coupling the conductive surface to a distal tip of the inner member. The procedure includes visualizing a distal tip of the outer member using an EAM system.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIGS. 2A-2B illustrate a mapping device with an internal electrode and an inner member in accordance with an embodiment of the present disclosure.

FIG. 2C Illustrates a cross-section of the device and inner member in FIG. 2A.

FIG. 3 illustrates a mapping device with an internal electrode and an inner member having a proximal contact in accordance with an embodiment of the present disclosure.

FIGS. 4A-4B illustrate a mapping device with an internal electrode and an inner member in accordance with an embodiment of the present disclosure.

FIG. 5 illustrates an arrangement of an inner member having a proximal contact in accordance with an embodiment of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

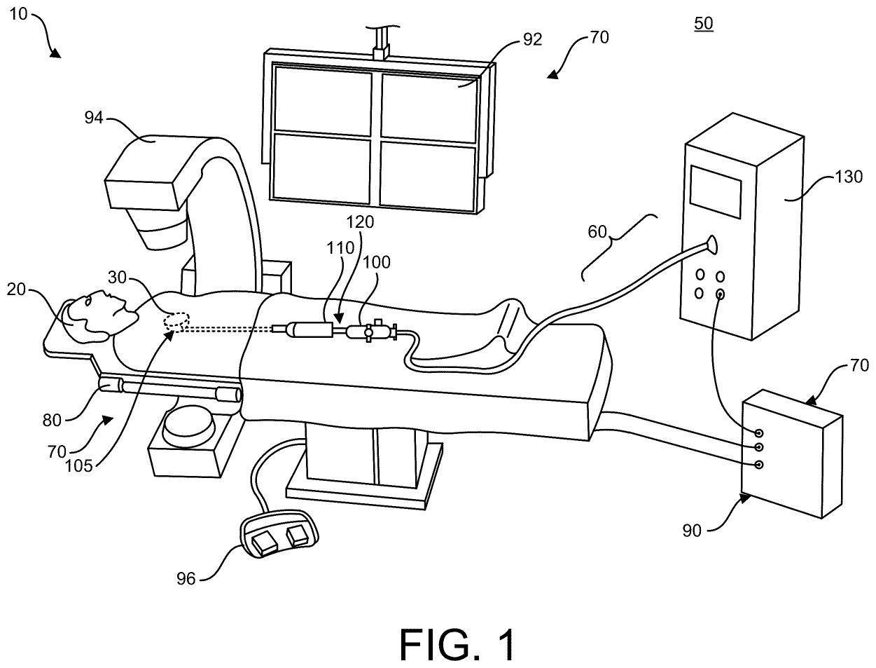

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an access system 60 and an auxiliary device, such as an EAM system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The access system 60 includes a dilator 100 having a proximal portion 102 and a distal portion 105, an introducer sheath 110, and a console 130. In some aspects, the distal portion 105 includes a tapered region. Additionally, the access system 60 includes various connecting elements, e.g., cables, umbilicals, and the like, that operate to functionally connect the components of the access system 60 to one another and to the components of the EAM system 70. This arrangement of connecting elements is not of critical importance to the present disclosure, and the skilled artisan will recognize that the various components described herein can be interconnected in a variety of ways.

In embodiments, the introducer sheath 110 is operable to provide a delivery conduit through which the dilator 100, in particular all or part of the distal portion 105 thereof, can be deployed to the specific target sites within the patient's heart 30. The dilator 100 is configured with a lumen such that a guiding device, for example a guide wire, or perforation device, for example an RF perforation device, can be inserted therein. In some aspects, a perforation device can be used to perform a transseptal crossing procedure within the heart 30.

The console 130 is configured to control functional aspects of the access system 60. In embodiments, the console 130 includes one or more controllers, microprocessors, and/or computers that execute code out of memory to control and/or perform the functional aspects of the access system 60. In embodiments, the memory can be part of the one or more controllers, microprocessors, and/or computers, and/or part of memory capacity accessible through a network, such as the world wide web. In embodiments, the console 130 can include pulse generator hardware, software and/or firmware configure to generate electrical pulses in predefined waveforms, which can be transmitted to electrodes positioned on the dilator 100, guiding device, or perforation device to generate electric fields sufficient to achieve the desired clinical effect, for example ablation of target tissue through irreversible electroporation. In embodiments, the console 130 can deliver the pulsed waveforms in a monopolar or bipolar mode of operation, as will be described in further detail herein.

The EAM system 70 is operable to track the location of the various functional components of the access system 60, and to generate high-fidelity three-dimensional anatomical and electro-anatomical maps of the cardiac chambers of interest. In embodiments, the EAM system 70 can be the RHYTHMIA™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

The EAM system 70 generates a localization field, via the field generator 80, to define a localization volume about the heart 30, and one or more location sensors or sensing elements on the tracked device(s), e.g., the dilator 100, generate an output that can be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances be enhanced by first creating a map of the electric field induced by the electric field generator within the anatomy of interest using a probe equipped with a magnetic location sensor, as is possible using the aforementioned RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the dilator 100 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map and/or the electro-anatomical map.

In order to reduce the number of cables or wires within the clinical setting 10, an outer member, for example dilator 100, may include an internal electrode. In addition to helping reduce the number of cables or wires, an electrode positioned inside the lumen of an elongated device addresses the issue of material transitions on the surface of the device. Having the electrode shrouded from the external environment allows the outer surface of the device to be constructed of a single uniform material. In some embodiments, an internal electrode may be achieved by incorporating a hypotube along at least a portion of the lumen. The distal tip of the hypotube effectively acts as conductor, which is capable of being visualized by the EAM system 70, whereas the remaining body of the hypotube is shrouded by the outer member effectively rendering it invisible impedance tracking.

The number of cables or wires may be reduced because the internal electrode can be electrically coupled to the EAM system 70 via an inner member (such as a guide wire or perforation device) inserted within the lumen. As such, the outer member does not require a dedicated connector and cable to communicate with the EAM system 70. The distal portion of the inner member can include exposed metal that will contact the internal electrode while a connector is attached the proximal portion of the inner member. This creates a circuit to the EAM system 70. In some embodiments, the inner member is completely insulated except for the exposed tip and a small section at the proximal end.

In use, as the inner member is advanced through the lumen of the outer member, the exposed tip makes contact with the internal electrode and the EAM system 70 shows the position of the distal tip of the internal electrode corresponding to the tip of the outer member. Once the inner member advances beyond the distal opening of the outer member, the EAM system 70 is no longer connected to the internal electrode and instead will track the position of the exposed tip of the inner member. In order for the EAM system 70 to visualize the distal tip of both the outer member and the inner member, the inner member can include a secondary contact point situated proximally from the tip. This contact point will maintain contact with the internal electrode after the exposed tip has advanced out of the outer member.

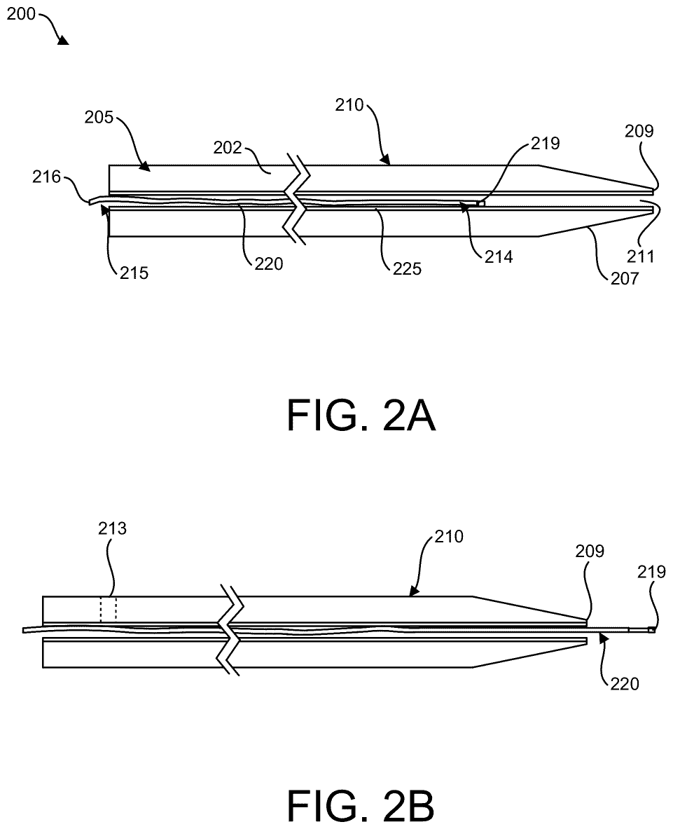

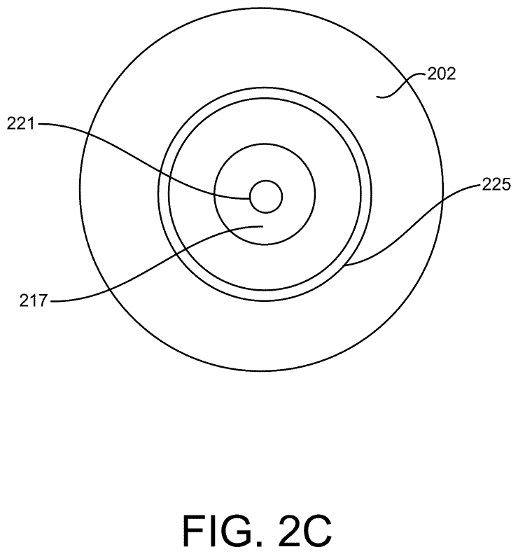

FIGS. 2A and 2B illustrate a system 200 including an outer member 210 and an inner member 220 in accordance with an embodiment of the present disclosure. The outer member 210 includes an elongate hollow body 202 having a proximal portion 205 and a distal portion 204. The distal portion 204 includes a tapered section 207 that reduces in diameter towards a distal tip 209.

The outer member 210 is an elongated device, such as a catheter, dilator, sheath, or other hollow elongate member. The outer member 210 defines a lumen 211 that extends from the proximal portion 205 to the distal tip 209. The lumen 211 is configured to allow for the introduction of fluids or an inner member 220 into and though the outer member 210. The proximal portion may include a hemostatic valve through which the fluids or inner member 220 pass to enter the lumen 211. In some embodiments, the fluids may include medications, contrast agents, or biological fluids. The proximal portion 205 may include a fitting, such as a luer connector, to which a syringe or other container for delivering fluid through the lumen 211 may removably connect. In various embodiments, the inner member 220 is a puncture device, such as a RF puncture wire or needle.

The lumen 211 is at least partially formed of an electrically conductive material 225 to allow current to flow through. The conductive material 225 forms a conductive surface along the lumen and creates an internal electrode. In some embodiments, the outer member 210 is formed of a hypotube, for example a stainless steel hypotube, having an electrically insulative coating. The inner surface of the hypotube forms the electrically conductive material 225. In other embodiments, the outer member 210 includes a conductive coating placed on the inner wall of the lumen 211. The elongate hollow body 202 may be formed of an insulative material that surrounds the conductive material 225.

The conductive material 225 extends towards the distal tip 209 and is positioned such that it is visible to impedance tracking by the EAM system 70. The conductive material 225 may extend from the proximal portion 205 to the distal tip 206. In some embodiments, the conductive material 225 extends beyond the distal tip 209. In some embodiments, the conductive material 225 is flush with the distal tip 209. In other embodiments, the conductive material 225 remains proximal from the distal tip, for example by a distance of less than 3 mm.

An inner member 220 is configured to traverse though the lumen 211 of the outer member 210. In some aspects, devices which are inserted into the lumen 211 can include perforation devices, such as mechanical or RF perforation devices, or guiding members such as guide wires. The inner member 220 includes a proximal portion 215 having a proximal end 216 and a distal portion 214 that includes a distal tip 219. In some embodiments, the distal portion 214 includes a preformed shape, such as a J shape or pigtail shape, which encourages the distal tip 219 to move away from a longitudinal axis of the outer member 210, such that the distal tip 219 contacts the conductive material 225 as the inner member 220 is advanced through the lumen 211.

The inner member 220 includes a conductive core at least partially surrounded by insulation. The proximal end 216 does not include insulation and is configured to connect to EAM system 70. Additionally, the distal tip 219 is uninsulated, and can form an electrode that can be used to pierce tissue or perform a measurement. In some embodiments, the distal tip 219 is used to deliver therapy to a target issue, for example, ablating tissue, puncturing the septum of a patient's heart to create a channel from one portion of the heart to another, or creating a channel from one anatomical structure to another. In one aspect, piercing the septum allows for a channel to be formed between the right atrium of the heart and the left atrium of the heart. In one aspect, the channel can provide access or communication from one anatomical structure to another for the advancement or delivery of medical devices, fluids, or therapeutic devices.

The uninsulated distal tip 219 is configured to electrically connect the conductive material 225 to the EAM system 70 as the inner member 220 traverses the lumen 211. This allows for different EAM visualizations of the inner member 220 and the outer member 210 depending on the situation. In FIG. 2A, the distal tip 219 of inner member 220 is located inside the lumen 211 and contacts the conductive material 225. As such, the inner member 220 acts as a conductor to transmit a signal received at the distal portion 204 where the conductive material 225 is exposed. In FIG. 2B, the inner member 220 protrudes from the lumen 211, disconnecting the conductive material 225 from the EAM system 70. As such, the EAM system now tracks the uninsulated distal tip 219 of the inner member 220.

Some embodiments include an access on the outer member 210 for electrically connecting the EAM system 70 directly to the conductive material 225. FIG. 2B illustrates a side port 213 that allows for access to the conductive material 225. An electrical conductor, such as a wire or lead, can be inserted through the side port 213 to couple the EAM system 70 with the conductive material 225. This allows for the exposed portion of the conductive material 225 to be utilized as an electrode without the need of inner member 220. In other embodiments, the access can take the form of a plug, patch, connector, clip, or other structure allowing for electrical coupling between the conductive material 225 and the EAM system 70, or other control system. In other embodiments, a raised feature associated with a clip, cap, or other connector can be used to penetrate through an outer layer surrounding the conductive material 225 in order to contact the conductive material 225. For example, the teeth associated with an alligator clip may be used to pierce an outer layer to electrically connect the EAM system 70 to the conductive material 225. Additionally, the raised threads of a screw-down connector may be used to slice through an outer layer to electrically connect the EAM system 70 to the conductive material 225.

FIG. 2C illustrates a cross-section of the outer member 210 and the inner member 220 in FIGS. 2A and 2B. The outer member 210 includes a conductive material 225 at least partially forming lumen 211. The elongate hollow body 202 is formed of an insulative material. The inner member 210 includes a coating or sheath of insulative material 217 surrounding a conductive core or wire 221.

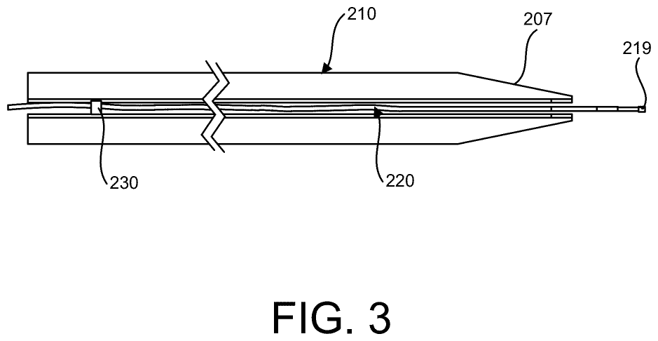

FIG. 3 illustrates an embodiment of an outer member 210 having an internal electrode and an inner member 220 having a proximal contact 230. The proximal contact 230 is an enlarged portion of the inner member 220 and is configured to make contact with the conductive material 225 and electrically couple the conductive material 225 to the EAM system 70. The proximal contact 230 is configured to at least contact the conductive material 225 after the distal tip 219 of the inner member 220 has protruded from the lumen 215. The proximal contact 230 is positioned far enough from the distal tip 219 such that it will not be extended out of the outer member 210 during use. The proximal contact 230 is positioned such that it contacts the conductive material 225 before the distal tip 219 exits the outer member 210. This allows the EAM system 70 to track both the outer member 210 distal tip 209 and the inner member 220 distal tip 219.

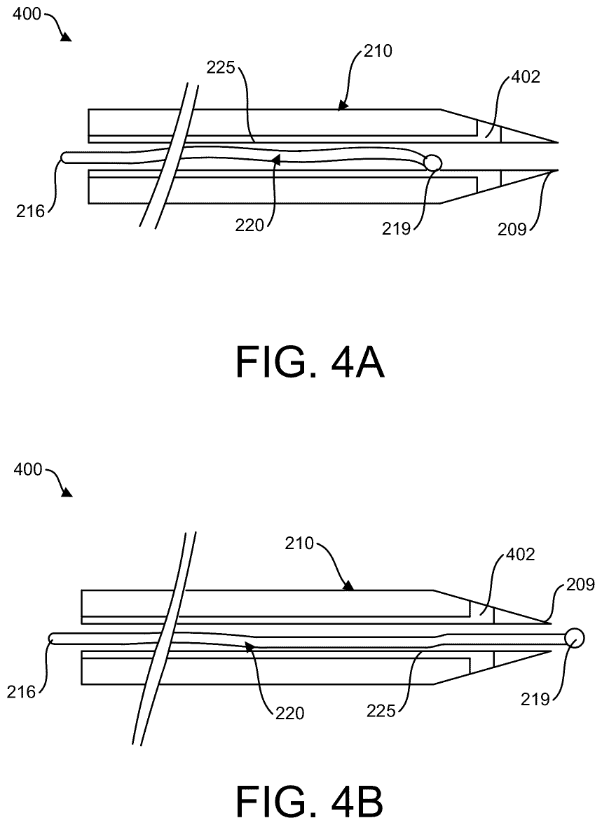

FIGS. 4A and 4B illustrate an embodiment of a system 400 having an outer member 220 and an inner member 210 similar to system 200. However, outer member 220 includes an electrode 402 on the distal portion 204. The electrode 402 is flush with an outer surface of the tapered section 207 and is electrically continuous with the conductive material 225. In some embodiments, the electrode 402 is integrally formed by the conductive surface 225. In other embodiments, the electrode 402 is formed of a first conductive material and the conductive surface 225 is formed of a second conductive material. In use, the EAM system 70 visualizes and displays the location of the electrode 402 when the distal tip 219 of the inner member is in electrical communication with the conductive surface 225 as discussed previously.

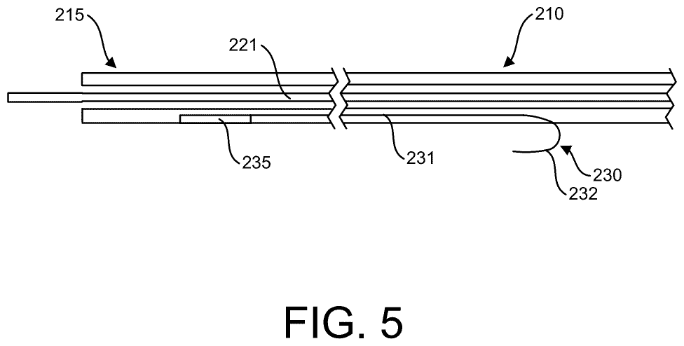

FIG. 5 illustrates an arrangement of an inner member 210 having a proximal contact 230 in accordance with an embodiment of the present disclosure. As illustrated in FIG. 5, the proximal contact 230 includes a retrograde wire. As the inner member 210 is advanced through the lumen 211, the retrograde wire 232 makes contract with the conductive surface 225 to electrically couple the conductive surface 225 to the EAM system 70. The retrograde wire 232 extends away from the inner member 232 and backwards in a proximal direction.

The retrograde wire 232 is coupled to a contact 235 via a conductor 231 for removably connecting to the EAM system 70 via a connector and/or cable. The retrograde wire 232, contact 235, and conductor 231 are insulated from conductive core 221. As discussed previously, the proximal contact 230 allows the EAM system 70 to visualize both the inner member 210 distal tip 219 and the outer member 220 distal portion once the distal tip 219 of the inner member 210 extends from the lumen 211.

In some embodiments, the outer member 210 or the inner member 220 includes a plurality of cuts machined into the wall, for example by laser cutting. The shape and positioning of the cuts can allow for a transition in flexibility from a proximal portion to distal portion. The cuts may include a broken spiral configuration or may be positioned substantially orthogonal to a longitudinal axis of the outer member 210 or the inner member 220. In some embodiments, there is a single cut that winds around an axis with a wider spacing between loops at the proximal portion and a larger spacing at the distal portion. The spacing and size of the cuts can be varied to achieve different flexibilities along the length of the outer member 210 or the inner member 220.

In some embodiments, the outer member 210 or the inner member 220 is formed of a shape memory material, such as a shape memory polymer or a shape memory metal. This would allow the outer member 210 or the inner member 220 to have a first shape at a first temperature, and a second shape at a second temperature. Shape transition may be initiated by inserting a heated solution into the outer member 210 or the inner member 220 or using electricity to heat a portion of the outer member 210 or the inner member 220.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A medical system for creating a channel between two anatomical structures, the system comprising:
an outer member having a proximal portion and a tapered distal portion, and a lumen extending from the proximal portion to the tapered distal portion, at least a portion of the lumen having an electrically conductive surface extending to a distal end of the tapered distal portion;
an inner member having a proximal portion and a distal portion, the inner member being configured to translate within the lumen and to deliver a therapy to a target tissue;
wherein the inner member distal portion includes a conductive distal tip configured to electrically couple with the electrically conductive surface of the outer member and the proximal portion is adapted to electrically couple the conductive distal tip to an auxiliary device.

2. The system of claim 1, wherein the outer member is a hypotube.

3. The system of claim 1, wherein the electrically conductive surface includes a coating.

4. The system of claim 1, wherein the electrically conductive surface extends from the outer member proximal portion to the distal end of the tapered distal portion.

5. The system of claim 1, wherein the therapy delivered by the inner member is to puncture the target tissue.

6. The system of claim 1, wherein the outer member includes a layer of insulation surrounding the electrically conductive surface.

7. The system of claim 1, wherein auxiliary device is an EAM system adapted to identify and display a location of the distal end.

8. The system of claim 1, wherein the outer member distal portion includes an electrode.

9. The system of claim 8, wherein the electrode is formed by the electrically conductive surface.

10. The system of claim 8, wherein the electrode is flush with an outer surface of the outer member.

11. The system of claim 1, wherein the inner member includes a proximal contact configured to electrically couple with the electrically conductive surface.

12. The system of claim 11, wherein the proximal contact includes an enlarged portion.

13. The system of claim 11, wherein the proximal contact includes a retrograde wire.

14. The system of claim 1, wherein the outer member distal portion includes a distal tip, and the electrically conductive surface extends beyond the distal tip.

15. A medical system for creating a channel between two anatomical structures, the system comprising:
an outer member having a proximal portion and a distal portion, and a lumen extending from the proximal portion to the tapered distal portion;
a conductive surface along at least a portion of the lumen, the conductive surface extending to the tapered distal portion;
a layer of insulation surrounding the conductive surface; and
an inner member having a proximal portion and a distal portion, the inner member being configured to translate within the lumen and to deliver a therapy to a target tissue;
wherein the inner member distal portion includes a distal tip configured to electrically couple with the conductive surface and a proximal end configured to electrically couple with an EAM system.

16. The system of claim 15, wherein the outer member comprises a hypotube.

17. The system of claim 15, wherein the conducive surface includes a coating.

18. The system of claim 15, wherein the inner member includes a proximal contact configured to electrically couple with the conductive surface.

19. The system of claim 18, wherein the proximal contact includes an enlarged portion or a retrograde wire.

20. A method of performing a medical procedure, comprising:
inserting an inner member into a lumen of an outer member, wherein the lumen includes a conductive surface;
electrically coupling the conductive surface to a distal tip of the inner member; and
visualizing a distal tip of the outer member using an EAM system.
